(19) United States
(12) Patent Application Publication
(10) Pub. No.: US 2012/0164079 A1
(43) Pub. Date: Jun. 28, 2012

(54) IMAGING OF BIOLOGICAL TISSUE UTILIZING NANOPARTICLES

(75) Inventor: Rakesh Sharma, Tallahassee, FL (US)

(73) Assignee: Florida State University Research Foundation, Tallahassee, FL (US)

(21) Appl. No.: 13/410,796

(22) Filed: Mar. 2, 2012

Related U.S. Application Data
(63) Continuation of application No. 12/396,281, filed on Mar. 2, 2009.
(60) Provisional application No. 61/032,716, filed on Feb. 29, 2008.

(51) Int. Cl. A61B 5/055; G01N 33/567

(52) U.S. Cl. 424/9.323; 424/9.34; 435/7.21

(57) ABSTRACT

Methods for NMR microimaging of biological tissue are provided. Polymeric nanoparticles are provided as a contrast agent to subject tissue to be imaged. Each polymeric nanoparticle may include an iron oxide core that exhibits superparamagnetic properties that enhance tissue imaging when placed within a magnetic field, an anionic surfacant applied to the core, a polymeric layer that encapsulates the core and the anionic surfacant, and an antibody attached to the polymeric layer to facilitate attachment to the subject tissue. Following the provision of the polymeric nanoparticles as a contrast agent to the subject tissue, the subject tissue may be images utilizing a twenty-one Tesla microimaging technique.

### CROSS REFERENCE TO RELATED APPLICATION

This application is a continuation of co-pending U.S. patent application Ser. No. 12/396,281, entitled “Nanoparticles that Facilitate Imaging of Biological Tissue and Methods of Forming the Same,” which was filed on Mar. 2, 2009, and which claims the benefit of U.S. Provisional Application No. 61/032,716, filed Feb. 29, 2008, and entitled “Systems and Methods for Biological Magnetic Resonance Imaging.” Both applications are incorporated by reference herein in their entirety.

### FIELD OF THE INVENTION

Embodiments of the invention relate generally to nanoparticles and more particularly to nanoparticles that may facilitate imaging of biological tissue.

### BACKGROUND OF THE INVENTION

When imaging tissue, such as biological tissue, a wide variety of imaging techniques are typically utilized, such as, magnetic resonance imaging (MRI). In some instances contrast agents are injected in tissue to enhance the appearance of the tissue. However, conventional contrast agents, such as iron oxide particles, may not adequately attach to tissue in which the agents are injected. This failure to adequately attach to tissue may lead to lower quality or lower resolution images. For example, an inadequate visualization of tissue protons may lead to lower quality of tissue contrast and lower resolution images.

Accordingly, there is a need for particles and/or nanoparticles that facilitate imaging of biological tissue. Further, there is a need for methods and/or techniques for forming particles and/or nanoparticles that facilitate imaging of biological tissue.

### BRIEF DESCRIPTION OF THE DRAWINGS

Having thus described the invention in general terms, reference will now be made to the accompanying drawings, which are not necessarily drawn to scale, and wherein:

FIG. 1 illustrates a flow diagram for forming nanoparticles, according to an example embodiment of the invention.

FIG. 2 illustrates an example scanning electron microscope (SEM) image of nanoparticles with different components of SPIOM complex in sketch diagram, according to an example embodiment of the invention.

FIG. 3 illustrates a flow diagram of one example method for utilizing nanoparticles in the imaging of biological tissue according to an illustrative embodiment of the invention.

FIG. 4 illustrates an example spectrometer, Rf insert with animal heart, and tuning console, that may be utilized for imaging according to an example embodiment of the invention.

FIG. 5 illustrates an example of pulse sequences and data acquisition using a PARAVISION 3.2 platform, according to an example embodiment of the invention.

FIG. 6 illustrates one example of images of a rat heart that may be obtained in accordance with an example embodiment of the invention.

FIG. 7A illustrates one example of a probabilistic atlas of a heart, according to an example embodiment of the invention.

FIG. 7B illustrates example images of a heart that may be generated in accordance with various embodiments of the invention.

FIG. 8 illustrates an example rat heart image that may be generated in accordance with various embodiments of the invention.

FIG. 9 illustrates another example rat heart image that may be generated in accordance with various embodiments of the invention.

FIGS. 10-13 illustrate various diffusion weighted images that may be generated in accordance with various embodiments of the invention.

FIG. 14 illustrates an example approach of segmentation of myocardial fibers using diffusion weighted MR images and coding of tensors in different directions, according to an example embodiment of the invention.

FIG. 15 illustrates an example Histology-MRI correlation by point by point match of MRI and histology digital image, according to an example embodiment of the invention.

FIG. 16 illustrates an example diagrammatic sketch showing clinical implications of SPIOM enhanced MR microimaging, according to an example embodiment of the invention.

### DETAILED DESCRIPTION OF THE INVENTION

Example embodiments of the invention now will be described more fully hereinafter with reference to the accompanying drawings, in which some, but not all embodiments of the invention are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Example embodiments of the invention may provide for the formation, preparation and/or synthesis of nanoparticles that may facilitate the imaging of biological tissue. In various embodiments, the nanoparticles may facilitate the preparation of high resolution images and rapid imaging of various biological matter, such as cardiac tissue. According to an example embodiment of the invention, nanoparticles as described herein may be injected or supplied to the biological tissues to be imaged according to various imaging techniques, examples of which are described herein. The nanoparticles may be injected or supplied to biological matter either in vivo or ex vivo, according to an example embodiment of the invention.

I. Formation of Nanoparticles

A wide variety of different nanoparticles may be utilized as desired in various embodiments of invention. A few example nanoparticles and the formation of those nanoparticles are discussed below. According to one example embodiment of the invention, the nanoparticles may include polymeric nanoparticles. The polymeric nanoparticles may be formed using a combination of sonication and/or non-solvent temperature induced crystallization to synthesize magnetic nanoparticles, and encapsulation by monodispersed polymers to achieve high yield.

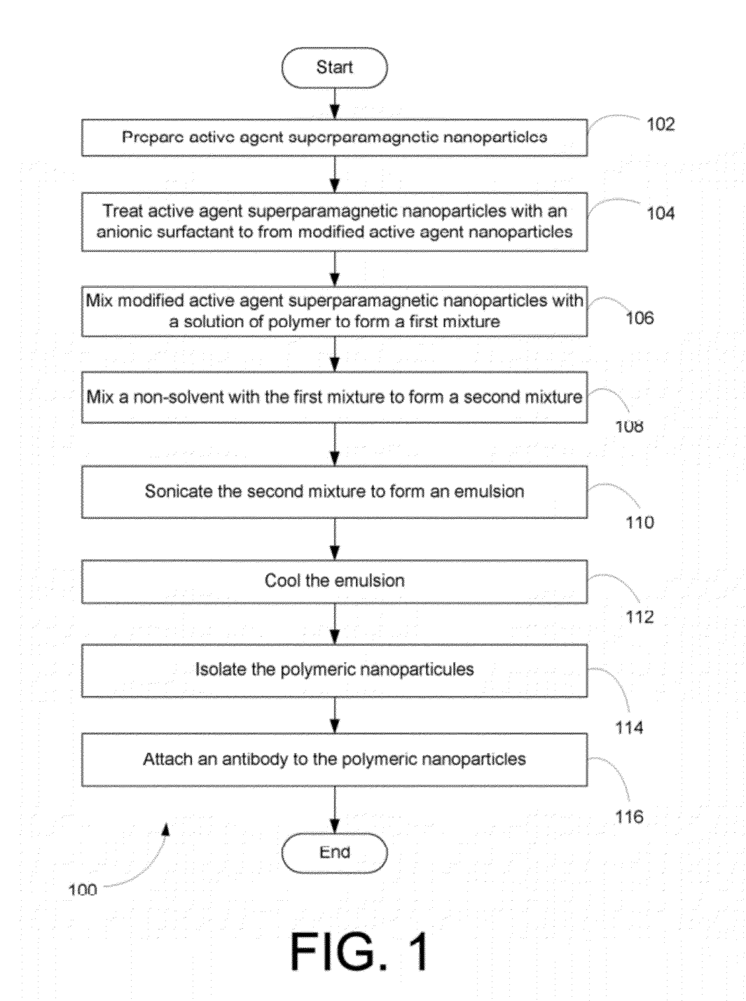

According to an example embodiment of the invention, a method 100 of forming nanoparticles is illustrated in the flow diagram of FIG. 1. The method 100 may include the preparation of nanoparticles that may be utilized in imaging of biological tissue. In certain embodiments of the invention, active agent superparamagnetic nanoparticles, such as active agent superparamagnetic iron ixode antimyoglobin (SPIOM) nanoparticles or antitroponin (SPIOT), may be utilized. During the formation of the nanoparticles, the nanoparticles may be subjected to a wide variety of sonication, solvent, non-solvent, and/or crystallization techniques as desired in various embodiments of the invention.

The method 100 may begin at block 102.

At block 102, active agent superparamagnetic nanoparticles may be prepared. One example active agent superparamagnetic nanoparticle is an iron oxide nanoparticle, although other superparamagnetic nanoparticles may be utilized as desired in various embodiments of the invention. For example, any nanoparticles that include relatively small ferromagnetic clusters that can randomly flip direction under thermal fluctuations or reduce nuclear magnetic resonance (NMR) relaxation constants may be utilized.

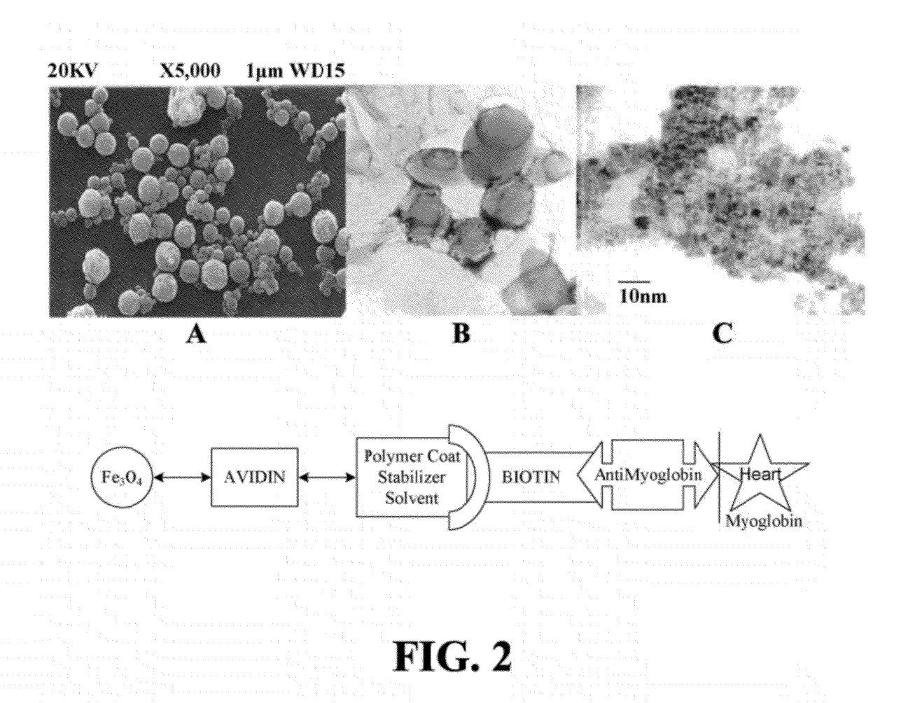

According to an example embodiment of the invention, active agent nanoparticles may be obtained in a desired core size depending on the nanoencapsulation, size, and/or surface charge of the particles. According to an example embodiment of the invention, one or more of the active agent superparamagnetic nanoparticles may have an average diameter between approximately 5 to approximately 100 nm as shown in FIG. 2. An example technique for producing iron oxide nanoparticles may involve co-precipitation and sonication to obtain active agent nanoparticles of average size between about 5 and about 10 nm for preparation of superparamagnetic antimyoglobin, antitroponin, or antimyosin particles. For example, iron-oxide nanocrystals may be mixed with 100 uL antimyoglobin mixed with 240 uM in 20 mM PBS pH 7.4. BioMAG avidin coated magnetic beads with Dynal® MyOne™ Streptavidin (Diameter-1.05 μm) (cat#650.01) from Dynal Biotech are one example.

With continued reference to FIG. 1, at block 104, the active agent superparamagnetic nanoparticles may be treated with an anionic surfactant to form modified active agent nanoparticles. For example, once active agent nanoparticles have been obtained, such as superparamagnetic iron-oxide nanoparticles or other active agent nanoparticles, the nanoparticles may be made susceptible to nanoencapsulation with monodispersed polymer by treating the particles with an anionic surfactant. In certain embodiments, the nanoparticles in a powder form may be added to an aqueous solution of an anionic surfactant, subjected to mixing conditions for a period of time, and then dried to remove the water so as to yield a dry powder comprising surface-modified superparamagnetic or active agent nanoparticles.

At block 106, the modified active agent superparamagnetic nanoparticles may be mixed with a solution of a polymer in a solvent at a first temperature. According to an example embodiment of the invention, the first temperature may be greater than the melting temperature of the polymer and less than the boiling point of the solvent so as to form a first mixture. Additionally, in certain embodiments, the mixing at block 106 may include the use of sonication or other similar methods and/or techniques.

At block 108, a non-solvent may be mixed with the first mixture to form a second mixture. According to an example embodiment of the invention, the non-solvent may be a non-solvent for the solvent and for the polymer and having a boiling point greater than the melting temperature of the polymer.

At block 110, the second mixture may be sonicated to form an emulsion. At block 112, the emulsion may be cooled to a second temperature and at a rate effective to precipitate polymeric nanoparticles (the polymer with the modified active agent superparamagnetic nanoparticles) dispersed therein.

At block 114, the polymeric nanoparticles may be isolated from the solvent and the non-solvent by any suitable isolation techniques as desired in various embodiments of the invention, such as filtering, reverse osmosis, etc.

At block 116, a suitable antibody may be attached to the polymeric nanoparticles. The antibody may facilitate the attachment of the polymeric nanoparticles to biological tissue that is imaged in accordance with various embodiments of the invention. A wide variety of different antibodies, such as antimyoglobin may be attached to a polymeric nanoparticle as desired in various embodiments of the invention. Additionally, as desired, a suitable protein-binding ligand may facilitate attachment of the antibody to the polymeric nanoparticles. As desired in various embodiments, a protein-binding ligand and/or an antibody may be selected or determined based at least in part on a type of tissue that the modified nanoparticles will be injected into for imaging purposes. For example, a tissue antigen specific antibody that facilitates the attachment of the nanoparticles to the tissue may be selected. As one example, the use of an antimyoglobin compound may facilitate attachment of nanoparticles to heart tissue. The antimyoglobin may attach to or pick up myoglobin molecules of the heart muscle to facilitate attachment of the nanoparticles to the heart tissue. For example, myoglobin molecules may be leaked from the heart muscle due to poor oxygen in the blood and/or heart muscle damage, and the antimyoglobin may attach to the myoglobin. In this regard, the nanoparticles may be attached to the heart tissue to facilitate imaging of the heart tissue.

The method 100 may end following block 116.

A wide variety of superparamagnetic nanoparticles may be utilized as desired in various embodiments of the invention. As one example, iron oxide nanoparticles may be utilized, and the iron oxide nanoparticles may be modified to form polymeric nanoparticles in accordance with various embodiments of the invention. An example technique for producing iron oxide nanoparticles may involve co-precipitation and sonication to obtain active agent nanoparticles of average size between about 5 and about 10 nm for preparation of superparamagnetic antimyoglobin particles. For example, iron-oxide nanocrystals may be mixed with 100 uL antimyoglobin mixed with 240 uM in 20 mM PBS pH 7.4. BioMAG avidin coated magnetic beads (Dynal® MyOne™ Streptavidin (Diameter-1.05 μm) (cat#650.01) from Dynal Biotech are one example.

Continuing with the example of the iron-oxide nanoparticles, the superparamagnetic may be made susceptible to nanoencapsulation with monodispersed polymer by treating the particles with an anionic surfactant. In one embodiment, the nanoparticles in a powder form may be added to an aqueous solution of an anionic surfactant, subjected to mixing conditions for a period of time, and then dried to remove the water so as to yield a dry powder comprising surface-modified superparamagnetic or active agent nanoparticles.

The surface-modified superparamagnetic nanoparticles may be sonicated into a solvent (e.g., a polyethylene solvent) to form a first mixture. The first mixture may then be subsequently mixed with a non-solvent to form a second mixture. The sonication of the first mixture (polymer/solvent/active agent particles) with the non-solvent may cause the formation of microspheres of the polymer with active iron-oxide particles in the second mixture.

During cooling, these microspheres may crystallize in the non-solvent by phase separation, according to an example embodiment of the invention. For example, where a polyethylene solution is utilized as the solvent discussed herein, the polyethylene solution may include a concentration of around 0.01 to around 0.1 weight/volume percentage (w/v %) (e.g., about 0.5 w/v %) dilution. Such a concentration may be utilized to support crystallization and the formation of nanoparticles of a desired size, according to an example embodiment of the invention. In this regard, nanoparticles of with a relatively small desired size may be formed.

In certain embodiments of the invention, ultrasonic mixing or ultrasonification may be utilized additionally or alternatively to the sonication described herein. According to an example embodiment of the invention, the sonication, ultrasonic mixing, and/or ultrasonication may use the application of acoustic energy to mix components together. Ultrasonic mixing at an amplitude between about 50% and about 60% for a period of time (e.g., around 30 seconds) at a temperature above the melting point of a utilized polymer may enable the formation of a homogeneous emulsion with dispersed phases of polymer and superparamagnetic material. According to an example embodiment of the invention, there may be a trade-off in that higher amplitudes may give smaller particles but generate significant amounts of unnecessary heat. At high temperature, an ultrasonication of solvent, non-solvent, polymer solution, and modified nanoparticles may cause the polymer to break into microdroplets of polymer solution, which form a microphase-separated system separating two liquid phases, according to an example embodiment of the invention. The subsequent cooling step may cool the emulsion to a second temperature at which polymer precipitates with the modified superparamagnetic nanospheres dispersed therein and crystallizes, in a non-solvent phase. The dispersed and crystallized polymer encapsulated superparamagnetic nanoparticles (e.g., iron oxide and polymer composite) may be isolated from the solvent and non-solvent by filtration or centrifugation.

According to an example embodiment of the invention, the composite nanospheres or nanoparticles may be coated with one or more protein binding ligands to give one or more functional properties as desired. Examples of protein binding ligands with iron oxide in the center include, but are not limited to, avidin, streptavidin, ferritin, etc. Examples of functional properties that may be achieved by coating a nanoparticle with a protein binding ligand include, but are not limited to, various enhancements to MRI visible functional properties of tissue, such as water proton spinning, water proton relaxation constants, water-fat proton contrast at an interface, and/or dynamic proton spins of tissue proteins, for example, myoglobin, troponin, myosin, etc. Other example functional properties include proton density changes in tissue and dynamic proton changes in functional proteins in tissue. For visualizing tissue functional changes, the nanoparticle functional properties of the ligands may restrict the core of the nanoparticles (e.g., an iron oxide core) and/or bind the polymer cage of the nanoparticles to facilitate imaging and the orientation of dynamic protons for imaging. A wide variety of coating techniques may be utilized as desired to coat a nanosphere or nanoparticle with a suitable ligand. Examples of suitable protein binding ligand coating techniques include, but are not limited to, using a fluidized bed method in which nanospheres or nanoparticles are suspending in a vertical column by air flow and sprayed with a suitable coating, engulfing a nanosphere or nanoparticle with a coating, and/or a technique in which ligands and nanoparticles are dispersed or dissolved in a polymer solution, mixed, suspended in a continuous phase, and the solvent is slowly evaporated. Other examples of coating include combining a polymer, such as polystyrene, polyethylmethylene, polypropylene, polyvinylalcohol, polyethyleneglycol, polyethylalcoholester, polyurethanes, polyamides, polycarbonates, polyalkenes, polyvinyl ethers, polyglycolides, cellulose ethers (e.g., hydroxy propyl cellulose, hydroxy propyl, methyl cellulose, hydroxy butyl cellulose, etc.), polyvinyl halids, and/or polylactic acid, with a suitable protein binding, such as, biotin, lectins, ferritin, albumin, etc.

Active agent nanoparticles that may be utilized in various embodiments of the invention may be stable across the range of temperatures that are utilized during a suitable nanoencapsulation process, such as that described above with reference to FIG. 1. Additionally, as desired in various embodiments, the active agent nanoparticles may be non-reactive to one or more solvents and/or non-solvents that are utilized in the nanoencapsulation process. Examples of active nanospheres that may be utilized as part of composites include drugs (i.e. therapeutic or prophylactic agents), diagnostic superparamagnetic agents (e.g., iron-oxide, gadolinium contrast agents), inorganic fertilizers, or inorganic pigments. Suitable superparamagnetic nanoparticles may include, for example, iron, nickel, cobalt, lanthanum, gadolinium, gold, zinc, manganese, and/or their alloys. In an example embodiment of the invention, iron oxide or maghemite (ΛFe_{2,3}) may be utilized for a nanoparticle to provide stability to oxidation. In another example embodiment of the invention, iron-neodymium-boron may be utilized for a nanoparticle as well. In an example embodiment of the invention, the superparamagnetic nanoparticles may include an average diameter between about 5 nm and about 10 nm.

In certain embodiments of the invention, suitable anionic surfactants may be utilized to treat active agent nanoparticles, as described above with reference to block 104 of FIG. 1. A wide variety of anionic surfactants may be utilized as desired including, for example, fatty acid salts such as sodium oleate. Other examples of suitable anionic surfactants may include, but are not limited to, sodium palmitate, sodium myristate, sodium stearate, and sodium dodecyl sulphate. It will be appreciated that while certain examples of anionic surfactants have been illustrated, other suitable anionic surfactants may be utilized without departing from embodiments of the invention.

In certain embodiments of the invention, active agent nanoparticles or modified active agent nanoparticles may be mixed with a solution of a polymer to form a first mixture, as described above with reference to block 106 of FIG. 1. A wide variety of suitable polymers may be utilized as desired in various embodiments of the invention. For example, a suitable polymer may include a crystalline polymer, such as a crystalline polymer that includes more than approximately sixty percent (60%) crystalline. Various polymers that are utilized may have different characteristics. For example, in one embodiment, an example polymer may have a boiling point of around 200° C., a melting point of around 150-180° C., and be a water resistant compound, suitable for temperature induced crystallization and/or a nanoencapsulation process.

In accordance with an example embodiment of the invention, a molecular weight of a utilized polymer may contribute to and/or determine the size of a composite nanoparticle that is formed. For example, a range of molecular weight between around one kilodalton (1 kDa) to around fifty kilodaltons (50 kDa) of the polymer may determine the size of a composite nanoparticle that is formed. For example, a polyethylene with an average molecular weight of 700 grams/mole or a polypropylene with an average molecular weight of 1,000 grams/mole may be useful in the nanoencapsulation process. Other examples of suitable polymers are polyamides, polycarbonates, polyalkenes, polyvinyl ethers, polyglycolides, cellulose ethers (e.g., hydroxy propyl cellulose, hydroxy propyl methyl cellulose, and hydroxy butyl cellulose), polyvinyl halides, polyglycolic acid, and polylactic acid. In one embodiment of the invention, the polymer may be a polyethylene polymer.

In certain embodiments of the invention, active agent nanoparticles and a polymer may be mixed in a solvent to form a first mixture, as described above with reference to block 106 of FIG. 1. The first mixture of active agent nanoparticles and polymer may be mixed with a non-solvent to form a second mixture, as described above with reference to block 108 of FIG. 1. According to an example embodiment of the invention, relatively high boiling solvents and/or non-solvents may enhance undercooling and/or speed up the crystallization process at a range between the melting temperature (at least 10° C.) and the crystallization temperature for a polymer. These effects may be due to a relatively high interfacial free energy associated with the basal plane of the crystallite to extract the ordered sequence to form a crystal. Additionally, in certain embodiments, the solvent and/or non-solvent may be relatively non-reactive with both the polymer and the active agent nanoparticles, such as active agent iron-oxide nanoparticles.

According to an example embodiment of the invention, the solvent may also be immiscible with the non-solvent at room temperature (e.g., about 20° to 27° C.). Other criteria for selecting the solvent may include the boiling temperature of the solvent. For example, a solvent with a boiling point at least approximately 10° C. higher than the melting temperature of the polymer may be utilized. In certain embodiments, the viscosity of the dilute solution in the solvent may be between about 2 and about 6 centipoise. Suitable non-polar solvents may include, but are not limited to, decalin, tetralin, toluene, dodecane, etc. Solvents that may be utilized with a polyethylene polymer may include, for example, decalin and octamethylcyclotetrasiloxane (OMCTS).

In an example embodiment of the invention, the non-solvent that is utilized may act well at a range between the boiling temperature and melting point and the temperature dependent miscibility associated with the solvent selected. A wide variety of non-solvents may be utilized as desired in various embodiments of the invention. For example, a suitable non-solvent that may be utilized for a polyethylene polymer is a tetraethylene glycol dimethyl ether (“tetraglyme”). This compound may be a polar organic compound utilized as a non-solvent.

In certain embodiments of the invention, one or more protein-binding ligands may be utilized to coat composite nanospheres or nanoparticles. Examples of suitable protein-binding ligands include, but are not limited to avidin, biotin, streptavidin, lectins, and/or any suitable biocompatible derivatives. In one example embodiment, iron oxide-avidin encaged in polyethylene nanoparticles can be formed and utilized in the preparation of antimyoglobin-biotin linked with avidin-polyethylene iron-oxide nanoparticles complexes. The avidin can act as a bridge that couples with polymeric nanoparticles modified with biotinylated antimyoglobin as shown in FIG. 2, which illustrates an example scanning electron microscope (SEM) image of nanoparticles with different components of SPIOM complex in sketch diagram. With reference to FIG. 2, iron-oxide nanocrystals may be mixed with antimyoglobin. Biotin-avidin may serve as a bridging link between the iron-oxide nanospheres and antimyoglobin. The nanoparticles may then be injected or otherwise brought into contact with biological tissue as desired, such as a heart muscle. The antimyoglobin antibody in the magnetic particle may attach to the polymer cage biotin on one side and outer free side with myoglobin terminal on the heart muscle, enabling the localized deposition of nanosphere due to antimyoglobin-myoglobin immunospecific binding in cardiac muscle.

A wide variety of different antibodies, such as antimyoglobin may be attached to a polymeric nanoparticle as desired in various embodiments of the invention. The antibodies may facilitate attachment of the polymeric nanoparticles to biological tissue that will be imaged. Additionally, as desired, a suitable protein-binding ligand may facilitate attachment of the antibody to the polymeric nanoparticles. As desired in various embodiments, a protein-binding ligand and/or an antibody may be selected or determined based at least in part on a type of tissue that the modified nanoparticles will be injected into for imaging purposes. For example, an antibody that facilitates the attachment of the nanoparticles to the tissue may be selected. As one example, the use of an antimyoglobin compound may facilitate attachment of nanoparticles to heart tissue.

In certain embodiments of the invention, the polymeric coated nanoparticles may be further encapsulated in a polymeric shell to provide additional functionality or a different functionality. For example, it may be desirable to ensure that the magnetic material is within the nanosphere. In addition, the ligand binding over polymer coating may further functionalize the iron-oxide particle. For example, a polyethylene styrene coated particle can be functionalized with a carboxyl group or hydroxyl group by copolymerizing the first layer with acrylates or phenolics, in order to couple the particle with a avidin protein. According to an example embodiment of coating (i.e. encapsulating) polyethylene magnetic nanoparticles, the coating polymer and the nanoparticles may be dispersed in a solvent for this polymer, such as a decalin and OMCTS solvent. The suitable classes of polymeric encapsulation materials may include polyesters, polyanhydrides, polystyrenes, and blends thereof.

In certain embodiments of the invention, the polymer coated nanoparticles may be substantially spherical, elliptical, or a mixture of the two. In an example range of 50 m to about 500 nm, the polymer coated nanoparticles may exhibit superparamagnetic behavior in microimaging applications. According to an example embodiment of the invention, 200 to 400 nm polymeric iron-oxide nanoparticles may include a polyethylene coat over maghemite (5-10 nm) nanoparticles and further include a avidin ligand coating adsorbed over the surface of the nanoparticles.

Nanoparticles that are formulated or created in accordance with various embodiments of the invention may be utilized in a wide variety of different applications, such as in imaging applications. The application of these particles in imaging may include real-time or substantially real time drug delivery monitoring or diagnostic imaging (e.g., for the delivery of contrast agents), magnetic separation processes, confocal laser scanning and fluorescent microscopes, magnetic resonance imaging (MRI), immunoassays, bioluminescent, photodynamic therapy, photoacoustic imaging, etc.

According to various embodiments of the invention, a wide variety of different polymeric nanospheres and/or nanoparticles may be formulated. As set for the above, one example of superparamagnetic nanoparticles that may be utilized are iron oxide nanoparticles. One example process for formulating polymeric nanoparticles from iron oxide nanoparticles will now be discussed in greater detail.

Iron oxide (γFe_{2O3}) particles having an average diameter range between about five (5) and ten (10) nanometers (nm) may be synthesized using an example three-step process of (i) co-precipitation of ferrous chloride and ferric chloride by sodium hydroxide, (ii) peptidization with nitric acid, and (iii) sonication. Ferrous chloride and ferric chloride may be mixed in a molar ration of approximately 1:2 in deionized water at a concentration of approximately 0.1 molar concentration (M) iron ions. After preparation, this solution may be mixed with a 10 M concentration solution of sodium hydroxide for coprecipitation with continuous stirring. Next, the solution with the precipitate may be stirred at a high speed for approximately one hour at about 20° C., and then heated to about 90° C. for approximately one hour with continuous stirring. The ultrafine magnetic particles obtained may be peptized by 2M nitric acid. Subsequently, the iron oxide dispersion may be sonicated for approximately 10 minutes at about 90° C. and at an amplitude of about 50%. The precipitate may then be washed repeatedly with deionized water and filtered and dried under a vacuum to yield fine iron oxide particles. The process set forth above for obtaining iron oxide particles is merely one example process for obtaining iron oxide particles. Other suitable processes, methods, and/or techniques may be utilized to obtain iron oxide particles or other superparamagnetic particles as desired in various embodiments of the invention.

Once the iron oxide particles are obtained, these particles may be modified with an anionic surfactant, such as sodium oleate, to facilitate and/or promote their attachment to a polymer, such as polyethylene. The modification may be carried out by mixing the iron oxide particles or powder with sodium oleate (at approximately 30% of the weight of the polymer) in water, and then stirring at a moderate speed for about 2 hours. The resulting mixture may then be dried by any suitable method or technique to remove the water, yielding a modified iron oxide powder useful in forming polyethylene composite particles.

As an example of forming a polyethylene composite particle, a dilute solution of a polymer, such as polyethylene wax, may be made. For example, a dilute solution of approximately 0.05% weight/volume polyethylene wax may be made using a solvent, such as decaline or OMCTS, at approximately 150° C. In one embodiment the polyethylene may be polyethylene wax with a weight average molecular weight (M_{W}) of approximately 700 grams/mole, such as a suitable polyethylene wax obtained from the Honeywell™ Corporation. Any amount of solvent may be utilized as desired, for example, approximately 10 milliliters of solvent. As desired, a quantity of the modified iron oxide powder may be added to this solution, perhaps at approximately 30% to approximately 50% of the weight of the polyethylene. The mixture may be sonicated at approximately 50% amplitude for about 30 seconds. Then, a volume of a non-solvent that is approximately equal to the volume of the solvent that was utilized (e.g., decaline, OMCTS) may be added to the mixture. For example, approximately 10 millileters of a non-solvent, such as tetraglyme (“TG”) (e.g., TG obtained from Sigma-Aldrich™), at approximately 150° C., may be added to the mixture, and the resulting second mixture may be sonicated at around 50% amplitude for about 30 seconds. The sonication of the second mixture may form an emulsion.

Next, the emulsion may be immediately cooled to about 0° C. by immersing a container holding the mixture, such as a scintillation vial, in ice water held at approximately 0° C. Within about three to four minutes of cooling, the emulsion may be transformed into a microphase separated system, which includes microdroplets of supercooled polyethylene wax solution and iron oxide dispersed in a continuous phase of non-solvent. Following the cooling of the emulsion, the polymeric nanoparticles may be isolated. For example, the emulsion may be warmed to room temperature (e.g., about 25 to about 27° C.) by removing the scintillation vial from the ice bath. Within about 45 minutes to about 1 hour, polyethylene particles, along with maghemite, may be found to be suspended in the emulsion. The emulsion may then be cooled to approximately −10° C. and maintained at this temperature for about half an hour in order to form a macrophase separated system.

After about a half hour, a thin reddish-brown layer may be observed at the interface of (i.e. between) a top layer of liquid (solvent) and a bottom layer of liquid (non-solvent). These top and bottom layers may then be extracted using any suitable extraction tools, for example, a micropipette and/or a syringe. The remaining solvent mixture (i.e. the reddish-brown layer), which contains the polyethylene/iron oxide particles, may then be centrifuged in a suitable centrifuge, such as a microcentrifuge, to isolate the particles from the remainder of the solvent mixture. The remaining solvent may then be removed by washing the particles with acetone. In this regard, the polyethylene/iron oxide particles or nanoparticles may be isolated.

The batch process described above for iron oxide nanoparticles may be repeated using various process parameters as desired in various embodiments of the invention. For example, six different batches of particles may be made using two solvents at two different speeds of sonication and with two different concentrations of polymers in each of the two solvents. In an example, embodiment, the second solvent (other than decalin) used may be octamethylcyclotetrasiloxane (OMCTS), such as OMCTS obtained from Dow Chemical Company™.

As desired in various embodiments of the invention, an appropriate amount of an avidin ligand may be dissolved in an adsorption buffer and utilized to form a monolayer around magnetic or superparamagnetic particles. For example, an avidin ligand may be dissolved in a sodium acetate/acetic acid with a pH of approximately five (5). The amount of protein utilized to form a monolayer around the magnetic nanoparticles may be determined and/or calculated using a wide variety of different techniques as desired in various embodiments. For example, the amount of protein utilized may be determined based on a desired amount for a diagnostic imaging test. According to an example embodiment of the invention, avidin may be used as ligand. A polyethylene magnetic particle suspension (e.g., a suspension in the same buffer that is approximately 10% solid) may be added to the protein solution and mixed gently for about one (1) to about two 2 hours. The suspension may then be incubated at room temperature for about 2 hours. The resulting mixture may then be centrifuged as desired.

According to an example embodiment of the invention, protein coupling efficiency may be measured for the composite particles using any number of desired methods and/or techniques. In one example, the supernatant was tested (using a BCA protein assay kit and a Turner spectrophotometer (SP 830) at a wavelength of about 562 nm) to determine the amount of bound proteins. A determination was made that about 30% amount of avidin was facilitated monolayer formation on polyethylene particles to coat the particles, leaving the remaining portion unabsorbed.

One challenge that may arise during the synthesis or formation of nanoparticles may be the desire to control the size of the nanoparticles. The high surface energy of the nanoparticles may contribute to this challenge. The interfacial tension applied to the modified nanoparticles in accordance with embodiments of the invention may facilitate reducing the surface area of the nanoparticles. In this regard, nanoparticles of a desired size and an acceptable size distribution may be synthesized. These nanoparticles may then be utilized in various imaging processes and/or techniques, such as magnetic resonance imaging (MRI), which is described in greater detail below.

II. Magnetic Resonance Imaging

Example embodiments of the invention may also provide for microimaging using superparamagnetic imaging nanoparticles. As desired, nanoparticles that are formulated in accordance with embodiments of the invention may be injected or otherwise provided to biological tissue, such as cardiac tissue, for use in imaging. The nanoparticles may be utilized in a wide variety of different imaging techniques, for example, magnetic resonance imaging. As one illustrative example discussed in greater detail below, iron oxide-polymer coated avidin-biotin bound antimyoglobin nanoparticles may be injected into heart tissue, such as a rat heart.

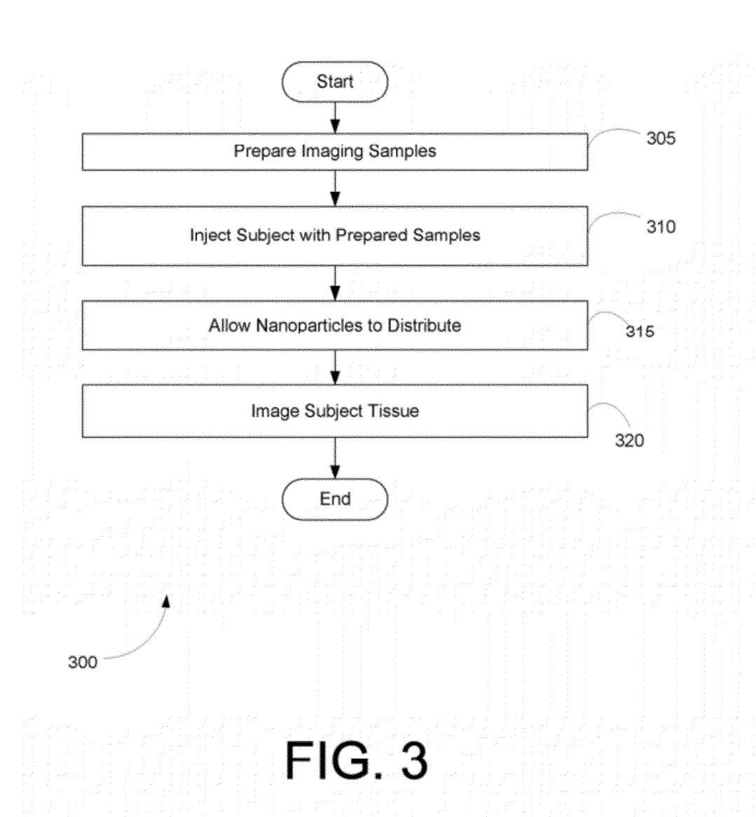

The nanoparticles-based microimaging may be utilized for a wide variety of different purposes as desired in various embodiments of the invention. A wide variety of different imaging techniques may be utilized as desired. These various imaging techniques may include different operations. A few examples of operations that may be included in an imaging technique, such as the imaging of a rat heart are described below with reference to FIG. 3.

FIG. 3 illustrates one example method 300 for utilizing nanoparticles in the imaging of biological tissue according to an illustrative embodiment of the invention. The method 300 may begin at block 305. At block 305, imaging samples may be prepared. One example of preparing suitable imaging samples may be preparing superparamagnetic nanoparticles (SPIOM) in a homogenous suspension. The amount of suspension to be utilized may be determined or calculated based on the weight of the animal or organism to be imaged. For example, the weight of the suspension may be calculated at approximately 10 miligrams/animal kilogram weight.

At block 310, the animal may be anesthetized or injected with the suspension or imaging samples. In one embodiment, a relatively slow anesthetization of an animal (e.g., rodent) or other subject may be utilized. For example, the SPIOM in suspension (10 mg/animal kg wt) may be injected into the animal or other subject via an intravenous (IV) insertion technique through a femoral vein route at a rate of approximately 2.5 mg/minute. The injection or insertion at this rate may facilitate a susceptibility effect.

At block 315, the nanoparticles may be allowed to distribute. For example, the injected animals or other subjects may be subject to an appropriate waiting time, perhaps at least approximately 20 minutes in an example embodiment, to allow a maximum or sufficient distribution of nanoparticles to subject tissue, such as cardiac mass or other subject tissue.

At block 320, the subject tissue may be imaged as desired in various embodiments of the invention. For example, the subject tissue may be imaged using an MRI technique. The injection of the SPIOM nanoparticles into the subject tissue may facilitate the enhancement of the imaging. The method 300 may end following block 320.

In some embodiments, the subject tissue, such as a rat heart may be excised or removed from the animal or subject. The excised tissue may be and perfused and oxygenated in a Kreb's Henseleit buffer, such as a buffer between approximately pH 7.2 and approximately pH 7.4 at approximately 37° C. (with approximately 95% O₂ and approximately 5% CO₂), in a hand made circulating tube system. According to an example embodiment of the invention, hearts may be arrested by a cardioplagic solution perfusion. After a heart is removed snugly and lifted from the myocardial cavity after clamping inferior vena cava, and all tributaries of the aorta and subclavian artery, the whole heart may be transferred into a Kreb's Henseleit buffer.

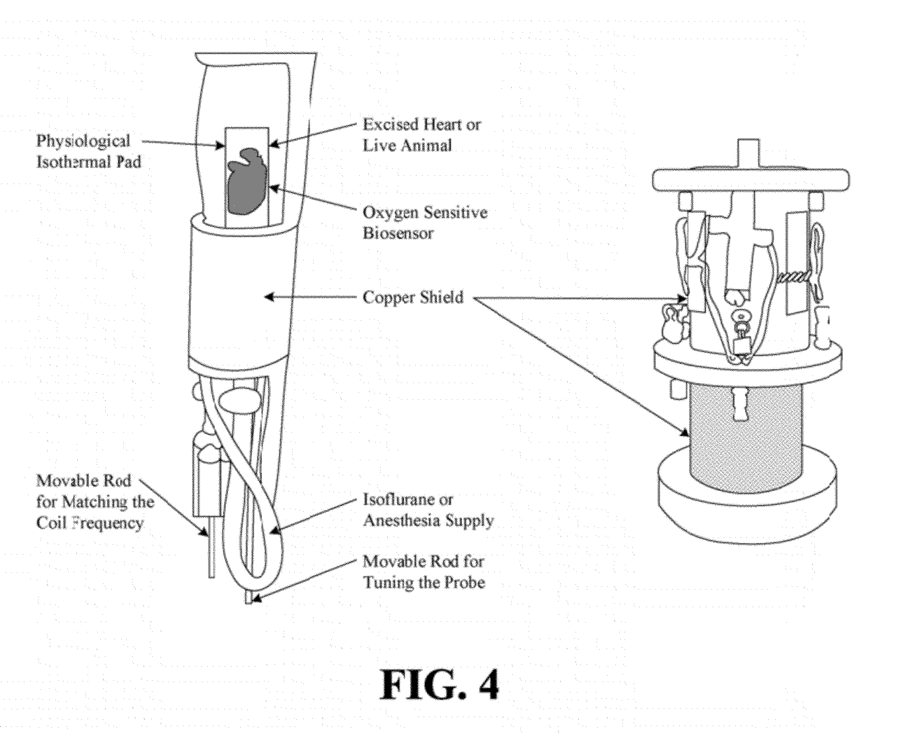

Continuing with the example of a rat heart that has been removed, the rat heart may be placed in a nuclear magnetic resonance (NMR) tube as desired. Additionally, a radio frequency (Rf) coil may be inserted into the NMR tube containing the rat heart, for example, by manual placement of the Rf coil insert with a pipe at a fixed height inside the magnet center of the K-space of the NMR tube, as illustrated in FIG. 4.

A magnetic imager may be tuned and/or matched as desired in various embodiments of the invention. In certain embodiments, a tuning (T) knob situated at or near the bottom of the magnet bore may be rotated to set Rf coil shimming by best cone tip at the center of an axis, such as an x-axis, on a monitor or other display associated with the magnetic imager. In other embodiments, the magnetic imager may be tuned based on bars associated with a tuning meter, such as bars in the center of the tuning meter. Additionally, the gradients may be matched by rotating capacitors situated in or otherwise associated with the Rf coil insert.

Additionally, as desired in certain embodiments, shimming may be utilized to calibrate a magnetic imager. For example, a central frequency may be calibrated by viewing an equilateral bell-shaped peak in center of an x-axis. For it, a gradient shimming display of x, y, and z in approximately 12 sets may be automatically optimized to obtain an equilateral single pulse with a minimum peak width.

In certain embodiments, a scan control and/or spectrometer control associated with a magnetic imager may be activated. For example, after shimming, an active control window or other user input device associated with the magnetic imager, such as a PARAVISION 3.2 active control window, may be used to select protocols and/or parameter settings as desired. A wide variety of protocols and/or parameters may be selected for an imager as desired in various embodiments. In one example embodiment, the selection or optimization of one or more microimaging parameters may include, for example, a GE Flow compensated (GEFC) slab selective at a flip angle=10 degree, sampling band width 100 MHz, acquisition time=2 minutes, and/or a 3D fast low angle shot (FLASH) pulse sequence at optimized TR=100 ms, TE=3.6 ms, FA=30, NEX=1, FOV=1.4×1.0 cm, matrix 1028×1028, in plane resolution=15 microns, acquisition time=12 seconds along short axis orientation to generate T2 weight while homogenizing the T1 saturation effects. In certain embodiments, a Multislice multiecho (MSME) spin echo sequence may be utilized by an imager at various parameters. Examples of parameters that may be utilized in one embodiment include TE/TR 15/1500 ms, NEX=1, FOV=0.9×1.7 cm, matrix=256×192 (for nanoparticles based dephasing on proton density weighting); matrix 1028×1028 (for nanoparticles based dephasing on proton density weighting). Examples of parameters that may be utilized in another embodiment include TE/TR 10/100 ms, NEX=1, FOV=0.9×1.7 cm, matrix=256×192 (for nanoparticles based dephasing on T1 weighting); TE/TR 10/100 ms, matrix 1028×1028 (for nanoparticles based dephasing on T1 weighting). In some embodiments, diffusion-sensitizing bipolar gradients in six non-colinear directions may be facilitated using TE=18 ms; TR=10000 ms; time interval between gradient pulses=5 ms; gradient pulse duration=0.5 ms, gradient factor=950 s/mm², b value of 950 s/mm², in-plane resolution of 35×35 micrometers, slice thickness=1 mm, slice gap=0.5 mm, and number of slices covering heart=7. Other example parameters may be utilized as desired in other embodiments of the invention. Additionally, the utilized parameters may be based at least in part on the imaging technology and/or imaging system that is utilized.

According to an aspect of the invention, the use of SPIOM particles may enhance the imaging of tissue, such as biological tissue. For example, the SPIOM may enhance the proton relaxation rate due to its dipolar relaxivity. For iron oxide SPIOM, the proton relaxation rate may be a function of the interaction between iron oxide and water molecules. In this regard, the SPIOM particles may enhance imaging. The images may show more data and or information. For example, when imaging a heart, more detailed information may be obtained for a ventricle wall, valves, chambers, and/or blood flow characteristics.

One example of in vivo relaxivities and susceptibility effects of SPIOM on an MRI signal will now be described. The nanoparticle SPIOM dephasing and MRI signal relationship can be shown as:

Signal=TEαexp^{(-TE/T2*)}, Eq. 1

where TE is echo delay time, and T2* is transverse relaxation constant due to susceptibility. T2* may be given by the following relationship:

1/T2*=1/T1+1/T2 Eq. 2

where 1/T2* is dephasing signal due to SPIOM induced myocardiac fiber specific field inhomogeneities measured by a GEFC sequence. The dephasing signal may be proportional to cubic nanoparticle radius. The susceptibility effect of SPIOM may enhance T2 relaxivity. The ratio of induced magnetization and applied magnetic field, such as a 21 Tesla applied magnetic field, may represent the susceptibility of a medium. Where the susceptibility increases, T2* may be understood as darkness or reduced MRI T2* intensity due to SPIOM induced local field gradients and an accelerated loss of phase coherence in spins contributing to the MRI signal. Additionally, at different concentrations (e.g., 100 μg/ml, 200 μg/ml, 400 μg/ml) of SPIOM, different T1 relaxation constants may be obtained and/or measured.

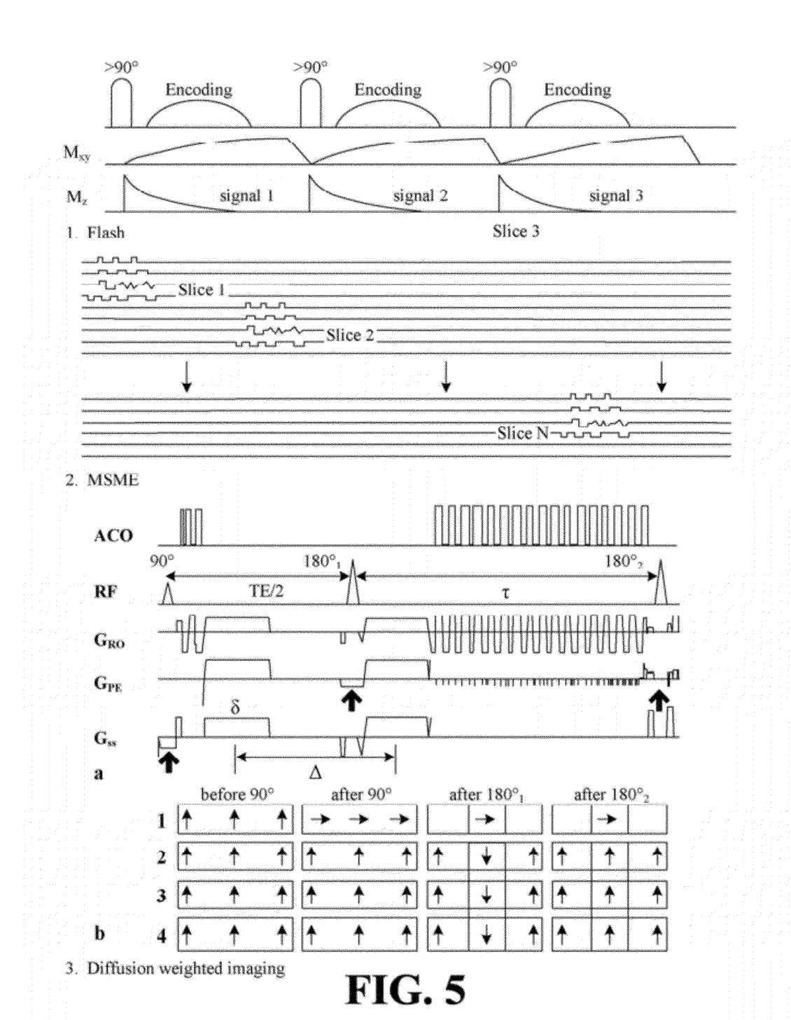

Once tissue is imaged, data may be acquired utilizing a wide variety of suitable techniques as desired in various embodiments of the invention. One or more images may be generated from the acquired data. FIG. 5 illustrates an example of pulse sequences and data acquisition using a PARAVISION 3.2 platform, according to an example embodiment of the invention. The spin echoes generate an NMR signal that may be converted into a time domain and a frequency domain by a Fourier Transform in both frequency and phase encoding directions. The display of the time domain may be changed by gradients in three directions of slice select or frequency encoded or phase encoded selection. The combination of gradients manipulation generates spatially encoded 2D or 3D or flow images. Further signal processing constructs an image inside magnet k-space.

According to an aspect of the invention, in vivo NMR microimaging may be used to calculate mean blood volumes during a cardiac cycle. Regional mean blood volume (MBV) maps of left ventricular myocardium may be computed pixel-by-pixel from steady state signals in sec⁻¹. For example, three (3) central short axis slices from each data set may be used for left ventricular region of interest (ROI) analysis. The left ventricle (LV) can be divided in 8 or more angular ranges on pre-SPIOM images at end-diastolic and end-systolic phases. The myocardium can be divided into three (3) transmural layers named as endocardial, mid myocardial and epicardial layers. The mid-wall septum may include a first 4 angular segments and a lateral wall may include the last 4 angular segments.

After image processing, the percentage (%) average MBV value can be calculated from MBV maps using average MBV in ROI of each specific layer, angular segment and cardiac points of end-diastolic (ED) and end-systolic (ES) phases. For example, the percentage average may be calculated by utilizing the following equation:

% average MBV=100%(MBV_{ED}−MBV_{ES})/MBV_{ED} Eq. 3

In example embodiments of the invention, nanoparticle enhanced contrast may include several quantitative possibilities and implications. For example, the injection of SPIOM may generate dark blood T1 images. The computed MBV_{ED} (during diastolic phase) and MBV_{ES} (during systolic phase) may show MBV maps by overlaying over pre-SPIOM images. As another example, pre-SPOIM and post-SPIOM images may be used to compute an MBV map of high short axis at five points and 8 angular segments at ED and ES.

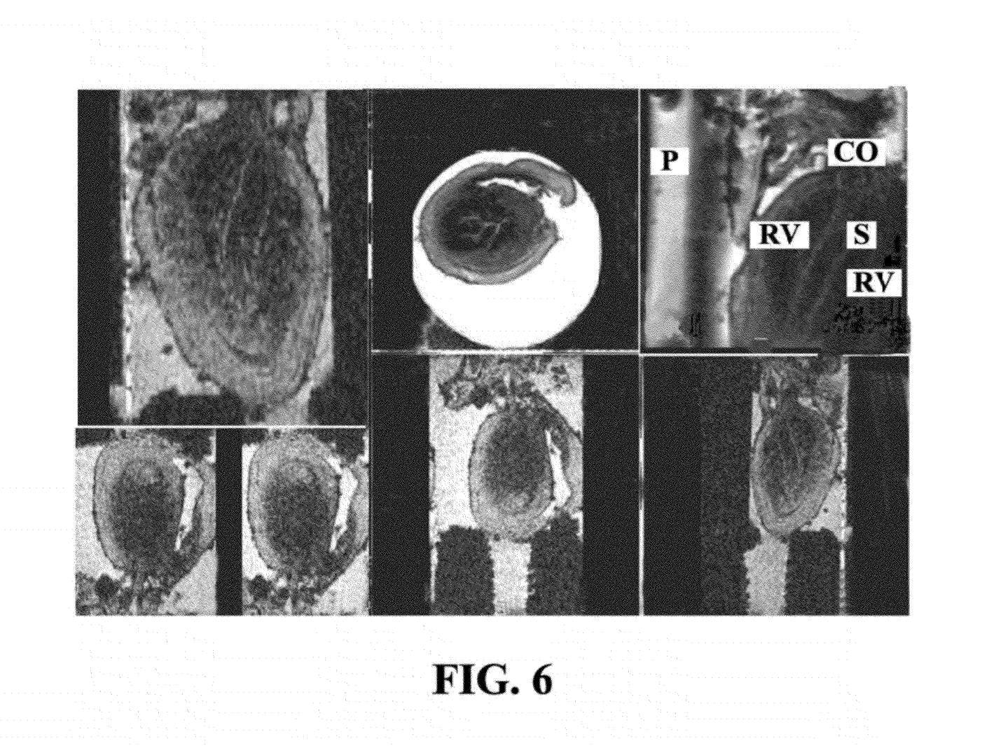

According to an aspect of the invention, generated images may be displayed by utilizing a wide variety of different techniques. For example, an image set display in a digital mode may show a pixel-by-pixel distribution of signal intensities on a gray scale in three axial, coronal and sagittal planes with T1 weighting, T2 weighting, and proton density weighting. Using an applied magnetic field of approximately 21 Tesla in association with a magnetic imager, such as an MRI, may facilitate the generation of diffusion tensor imaging weighted (DTI) images with diffusion-sensitizing bipolar gradients in six non-colinear directions displayed as tensor maps. FIG. 6 illustrates one example 605 of images of a rat heart that may be obtained. As desired, a suitable three-dimensional (3D) reconstruction, such as a 3D reconstruction mode using an ImagePro 3D reconstruction program, may be utilized to generate a 3D set of fast low angle shot (FLASH) images 610 to display the heart images in three planes. (See, for example, FIG. 6). In certain embodiments, the use of gradient echo pulse sequence techniques, such as FLASH techniques, may facilitate the generation of 3D images in various angular spatial directions, also referred to as 4D images.

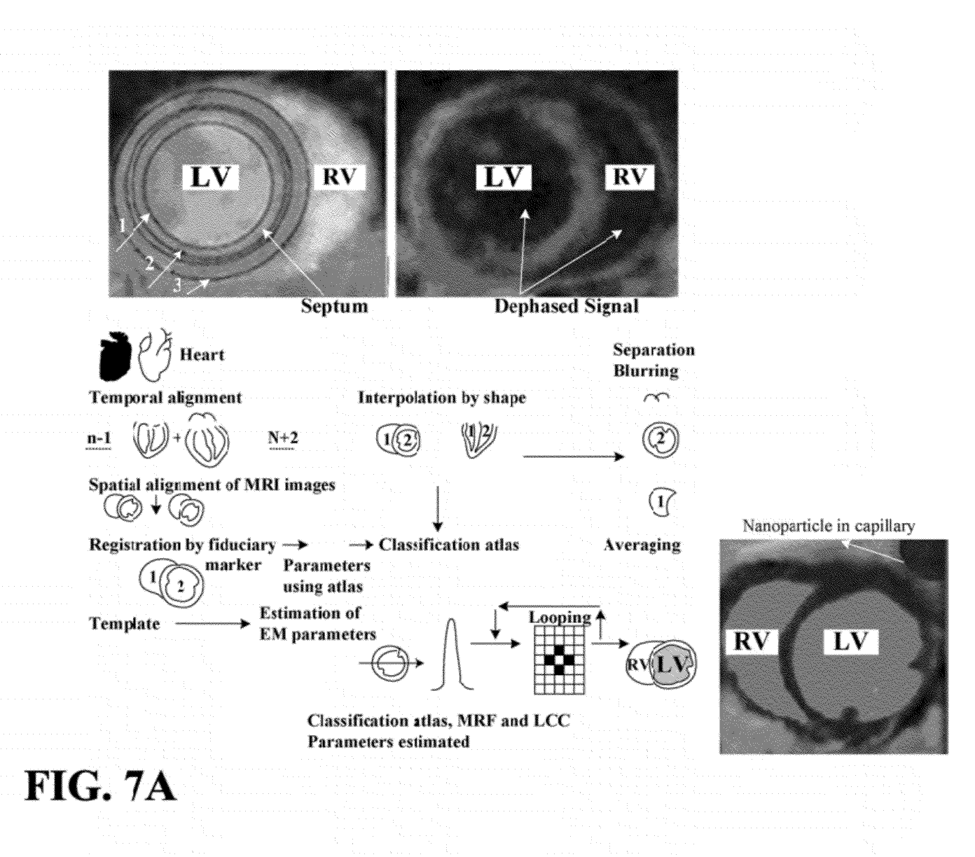

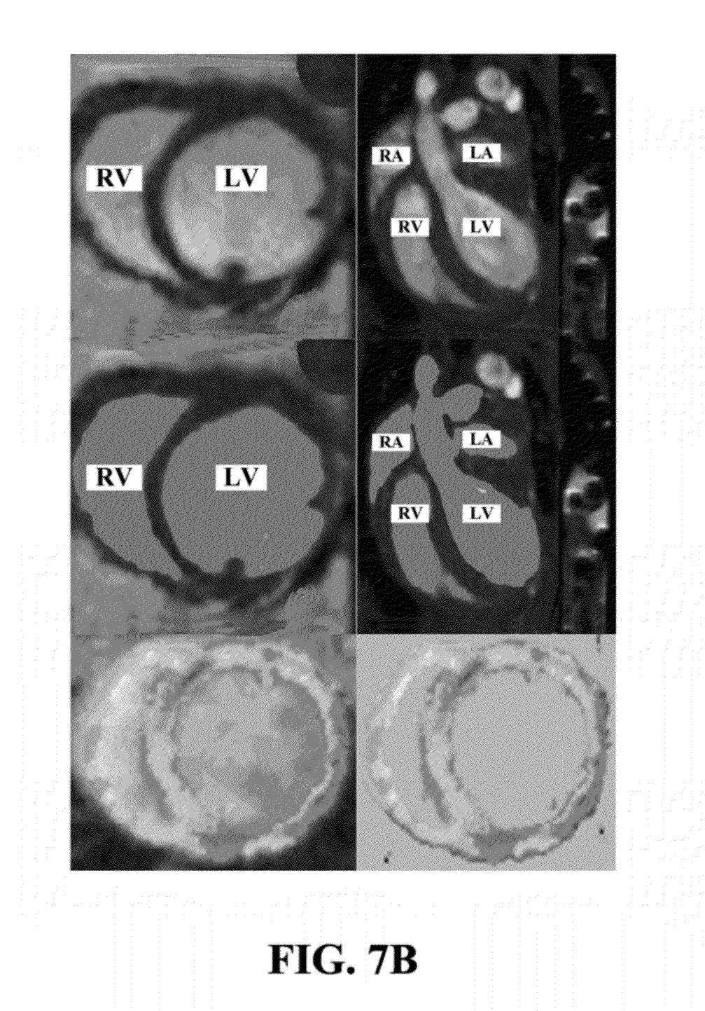

In certain embodiments, a cardiac segmentation may be based on an estimation of maximum likelihood (EM) algorithm and may be used to perform the construction of a probabilistic atlas. An EM algorithm may be an iterative method utilized to estimate a maximum likelihood for the observed data by estimating missing data and maximizing a likelihood for the estimated complete data. The MR microimaging observed signal intensities and missing data may be accomplished with the parameters that describe the mean and variance of each anatomic structure by a Gaussian distribution. (See, for example, FIG. 7A).

One illustration of the construction of a probabilistic atlas of a heart is illustrated in FIG. 7A. As shown in FIG. 7A, the cardiac atlas may be constructed and it may have multiple components, such as, spatial and temporally varying four dimensional (4D) probabilistic maps of four heart anatomic structures (LV, RV, myocardium, background). A 4D image may be a 3D image that may be rotated and/or moved in different directions. A priori knowledge of these structures may provide coding of cardiac anatomy and its spatial and temporal variability. Another example component is a template created by averaging the intensities of the MR image to create the cardiac atlas.

In certain embodiments, probabilistic maps may be utilized to automate the estimation of initial mean and variation parameters for each structure of an image. These maps may also provide spatial and temporal variability of different anatomic structures using a priori knowledge. For example, images may be manually segmented, sample-based and interpolated to get isotropic resolution. One image can be chosen as a reference and other images may be registered by an affini method to put all images in an appropriate or correct position, size and orientation alignment. The probabilistic map may be calculated by blurring the segmented image from each cardiac structure with a standard deviation of Gaussian kernel equal to 2, and by using subsequent averaging of the images. The final probabilistic atlas possibly may have a volume of 256×256×100 voxels. FIG. 7A shows the maps of a left ventricle, right ventricle and a myocardium.

In certain embodiments, a 3D template of one or more images may be calculated by normalizing and averaging the intensities of several images, after spatial alignment to a reference image. The intensity template may facilitate aligning the cardiac atlas with the images before their segmentation, as shown in FIG. 7A.

In embodiments that use a semi-automated segmentation approach for a heart image, a 3D intensity template may be registered to the left ventricle image (before its segmentation) to generate transformation in alignment with a probabilistic atlas. For temporal alignment, a mask may be generated for each tissue class (LV, RV, myocardium and background) in an atlas with at least 50% probability of belonging to each class. Each mask may calculate a mean and a variance of each class using the other images to perform a first classification. The first classification may include the highest probability for a background voxel at a particular position ‘i’. However, an image may show misclassified regions (vessels similar to myocardium). The largest connected component (LCC) of each structure may serve as a global connectivity filter and each LCC may be utilized to remove the false class of small unwanted structures. (See, for example, FIGS. 6A, 6B). This procedure may be repeated until maximum iterations are reached with complete coverage. The EM parameters in subsequent iterations can be subtracted again and again to minimize the difference (>0.01) until the procedure is stopped.

FIG. 7B illustrates example images of a heart that may be generated in accordance with various embodiments of the invention. With reference to FIG. 7B, the top images may illustrate post-nanoparticle enhanced mid cardiac territories showing distinct wall boundaries in an axial plane (left panel) and in a coronal plane (right panel). The middle images may illustrate supervised segmentation of post-nanoparticle enhanced mid cardiac territories showing the delineation of cardiac chambers by thresholding. The bottom images may illustrates color coded feature analysis of a wall by using a trained data set with a distinct color coding matrix by a 4D Expectation Maximization method, according to an example embodiment of the invention.

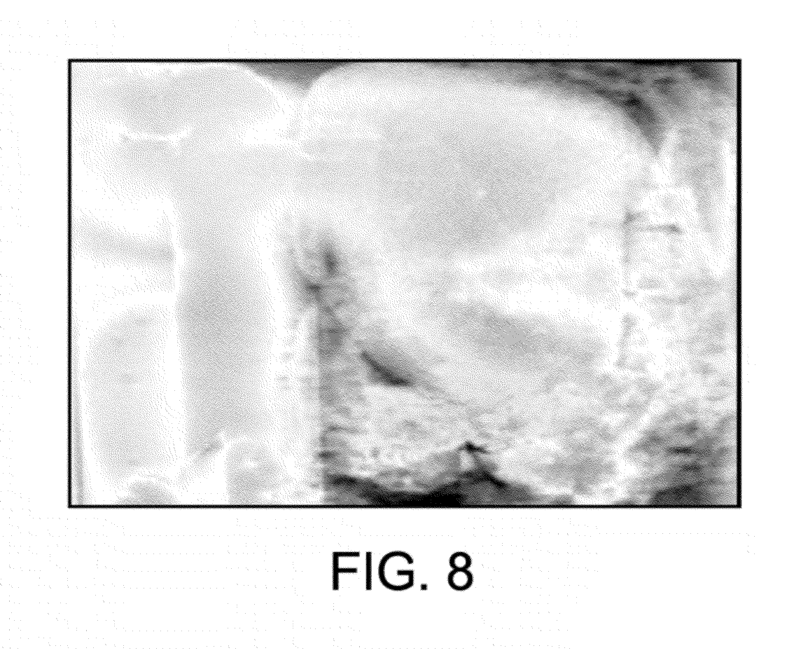

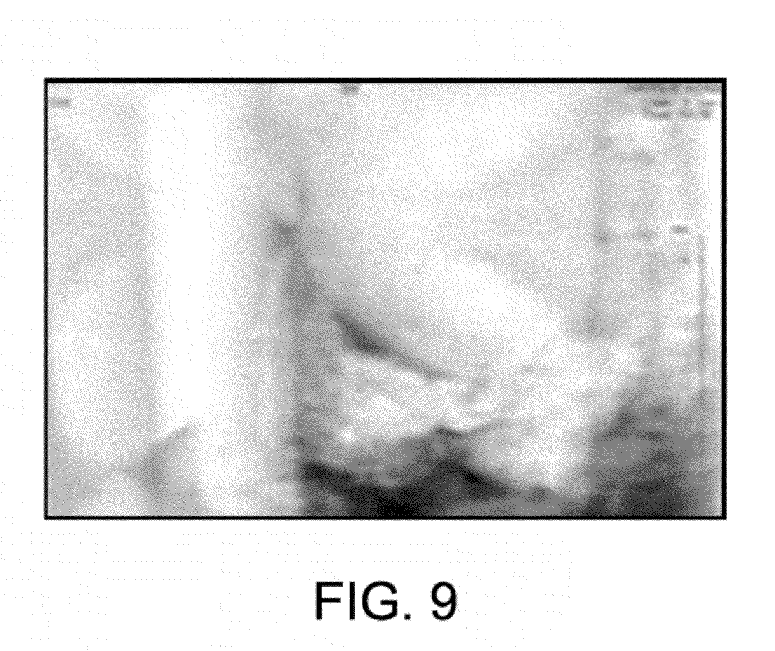

In certain embodiments, the proton density weighted and T1 weighted MSME images may display smooth cardiac mass with relatively little noise. For example, in FIG. 8, an example rat heart is shown using a multislice multiecho (MSME) pulse sequence using certain parameters associated with a 21 Tesla imaging device. Example parameters may include TE/TR of 15/1500 ms, NEX of 1, matrix of 128×128, and FOV of 0.9×1.7 cm. Of note in FIG. 8 is the distinct capillary filled with nanoparticles showing poor dephasing on T1 weighting. Similarly, in FIG. 9, an example rat heart is shown using a multislice multiecho (MSME) pulse sequence using parameters including: TE/TR of 15/1500 ms, NEX of 1, matrix of 128×128, FOV of 0.9×1.7 cm. Of note in FIG. 9 is the distinct capillary filled with nanoparticles showing dephasing on proton density weighting.

In various embodiments of the invention, the image processing of diffusion weighted images may be used for effective diffusion tensor (D_{eff}), diffusion characteristics, myocardial fiber orientation, and/or Laminar fiber sheet orientation. For example, FIGS. 9-12 illustrate various diffusion weighted images that may be generated in accordance with various embodiments of the invention.

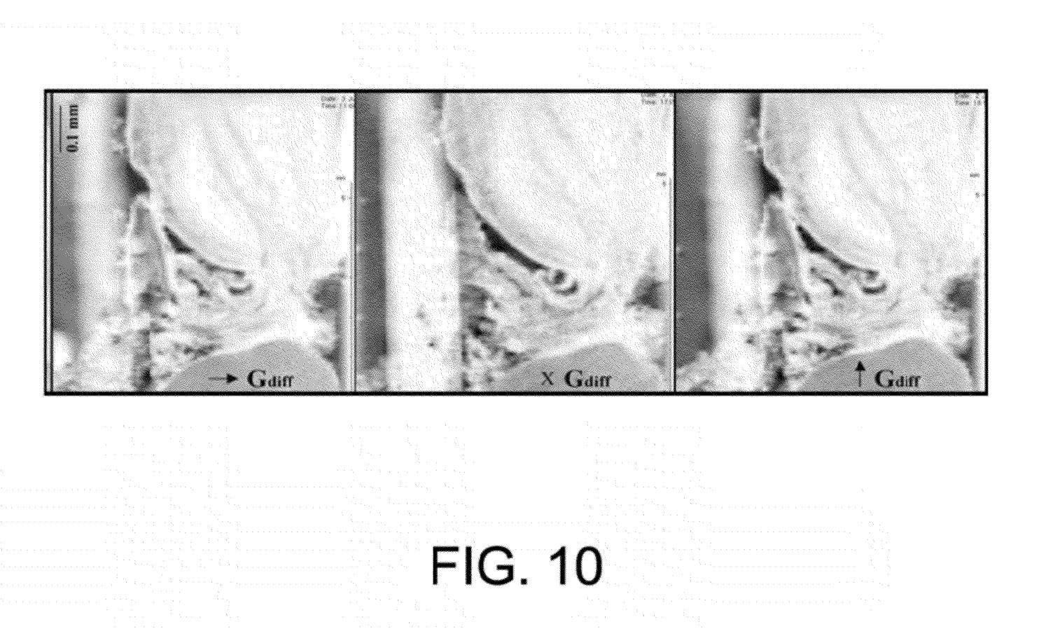

FIG. 10 illustrates example DW images of an isolated rat heart with a diffusion encoding gradient placed along the read (A), slice (B), and phase (C) directions (the gradient direction is indicated by an arrow, or an “x” for through-plane). The image shown in FIG. 10 may use various imaging parameters as desired, such as a TR/TE of 18.4/10000 ms, a b-value of 350 s/mm², a spatial resolution of 35 μm in-plane, and/or a slice thickness of 0.1 mm.

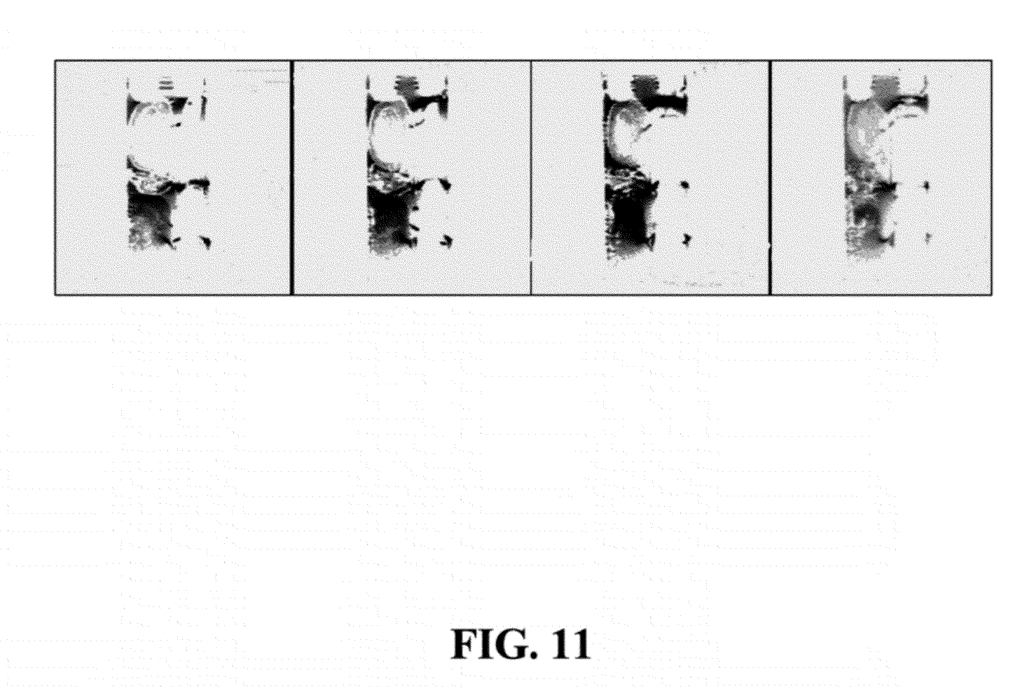

FIG. 11 illustrates example DW images of an isolated rat heart with a diffusion encoding DTI standard sequence and gradients placed along the read (A), slice (B), and phase (C) directions (the gradient direction is indicated by an arrow, or an “x” for through-plane). The image shown in FIG. 11 may use various imaging parameters as desired, such as a TE/TR of 18.4/10000 ms, a NEX of 1, a b-value of 350 s/mm², a FOV of 2.0/2.3 cm, a spatial resolution of 35 μm in-plane, a number of slices of 20, and/or a slice thickness of 0.1 mm.

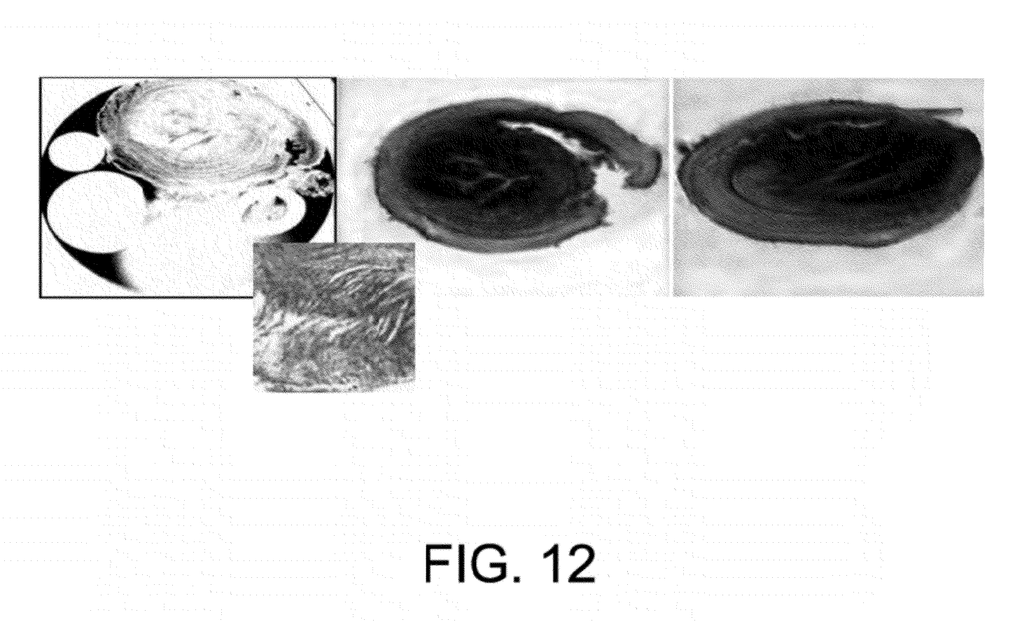

FIG. 12 illustrates example images in which in the left image, the excised rat heart after nanoparticle injection was imaged by a proton density weighted sequence at parameters that may include a TE/TR of 15/1500 ms, a NEX of 4, a FOV of 1.0×1.0 cm, a matrix of 256×256, a cycle of ¾, and/or a scan time of about 2 minutes. As shown in FIG. 12, there may be distinct layers of ventricle wall at the mid-ventricle level. The wall micro details are shown in the insert with an arrow. The capillary filled with nanoparticles may appear as a relatively dark color due to a dephasing effect on images. The dephasing effect may be concentration dependent as shown in Capillary A (200 μg/ml), Capillary B (400 μg/ml), and Capillary C (1000 μg/ml). A dephased signal intensity in the order of A<B<C is shown. The capillary C shows a relatively darkest signal and that diffused outside the boundary of capillary. In the middle image, a representative image was acquired by a FLASH_triplot pulse sequence, according to an example embodiment of the invention. The resolution power of microimaging illustrates distinct layers of ventricle walls in an axial plane. Sample parameters that may be utilized to form this image include a TR of 100 ms, a TE of 3.6 ms, a FA of 30, a NEX of 1, a FOV of 1.4×1.0 cm, a matrix of 1028×1028, an in plane resolution of 15 microns, and an acquisition time of 12 seconds. The right image was acquired with a GE Flow compensated (GEFC) slab selective at a flip angle of 10 degrees, a sampling band width of 100 MHz, and an acquisition time of 15 seconds. Of note is the rapid data acquisition and sufficient contrast of cardiac wall layers visible at midventricle level in axial plane.

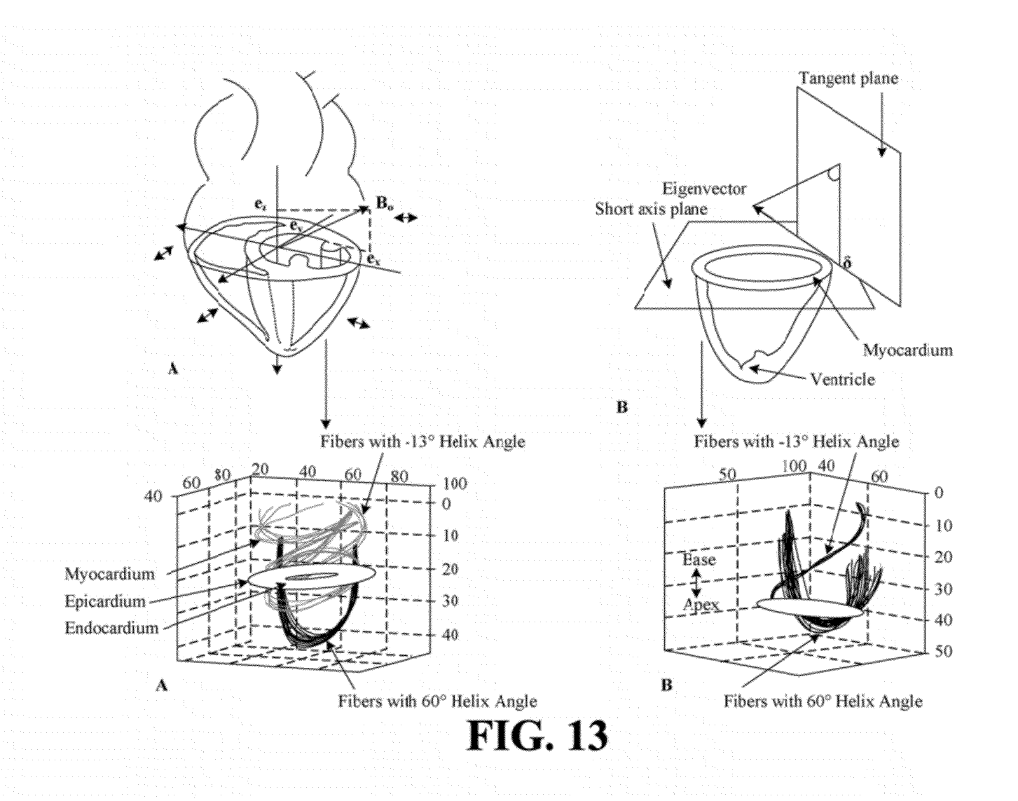

In FIG. 13, (at the top) the sketch illustrates directions of eigenvectors with arrows at the level of mid-ventricle (panel A) and three different planes with resultant eigenvectors shown with arrows. The cardiac fiber orientation (at bottom) is shown tracked from a slice located at the middle of the ventricle (shown in the plane) using two different helix angles of negative thirteen (−13) degrees (may be blue colored fibers) and sixty (60) degrees (may be red colored fibers). The other area (green area) shows left ventricle myocardium. In panel B, a green area shows an apex region and shows blue and red fibers at different said helix angles.

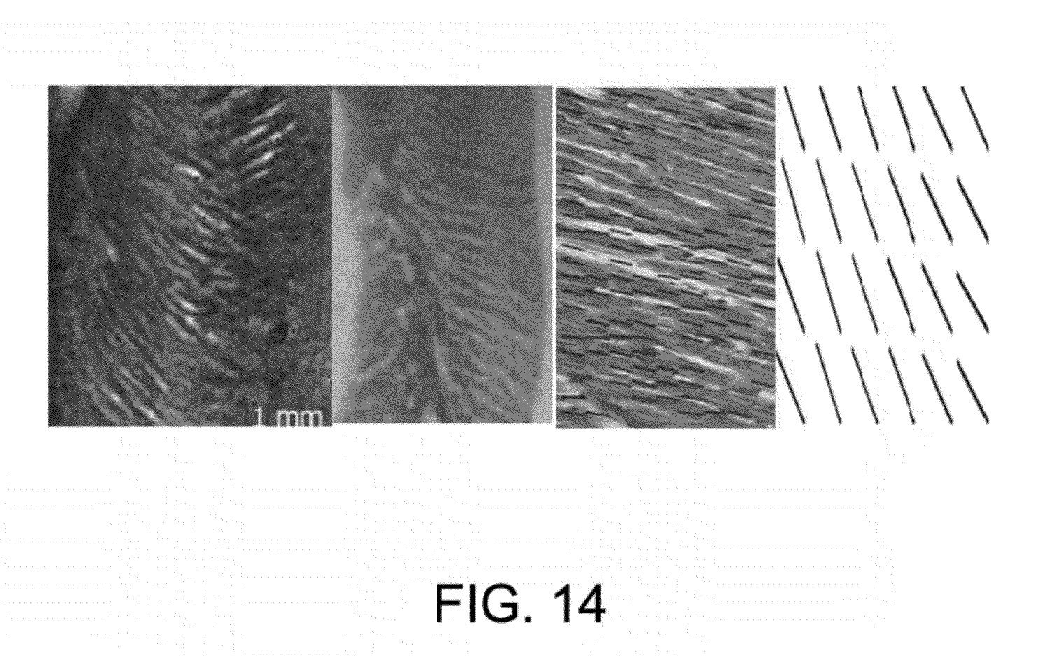

In certain embodiments, a quantitative characterization of contraction related fiber orientation at apex, midventricle, apex from primary eigenvector, and sheet orientation by secondary and tertiary eigenvectors may offer an evaluation of radial myofiber shortening. The transmural distribution of myofiber helix angles (αₕ), transverse angle (αₜ), and sheet angle (βₛ) in myofibers at endocardium and epicardium locations can predict geometrical changes in both the sheet and fiber orientation as a possible mechanism of radial wall thickening or myofiber shortening in a pulsating heart, as shown in FIG. 13. In an excised heart represented end-diastole phase, each slice data may be analyzed at anterior, lateral, inferior, and septal regions at approximately 20 degree sectors to calculate a transmural change of fiber orientation or through wall difference=Δαₕ=α_{h(endocardium)}−α_{h(epicardium)}. For example, FIG. 14 illustrates an example approach of segmentation of myocardial fibers using diffusion weighted MR images and coding of tensors in different directions, according to an example embodiment of the invention.

As desired in certain embodiments, a tissue mass, such as a cardiac mass may be delineated and measured. For delineation of a cardiac feature mass, the cardiac featured may be extracted out by manual delineation including the use of various methods of edge detection or thresholding. For measuring deformity, curves of cardiac structures texture analysis may be used as desired. Texture analysis may measure delineation of a margin of possible wall deformity or subtle curvatures by using an occurrence matrix (e.g., a vector of two voxel intensities) to evaluate contrast, correlation, homogeneity, and/or entropy. This matrix may specify scale and orientation in a texture anisotropy analysis. Other approaches include the manipulation of a gradient density matrix by convolution to calculate an intensity gradient vector in cylindrical polar coordinates.

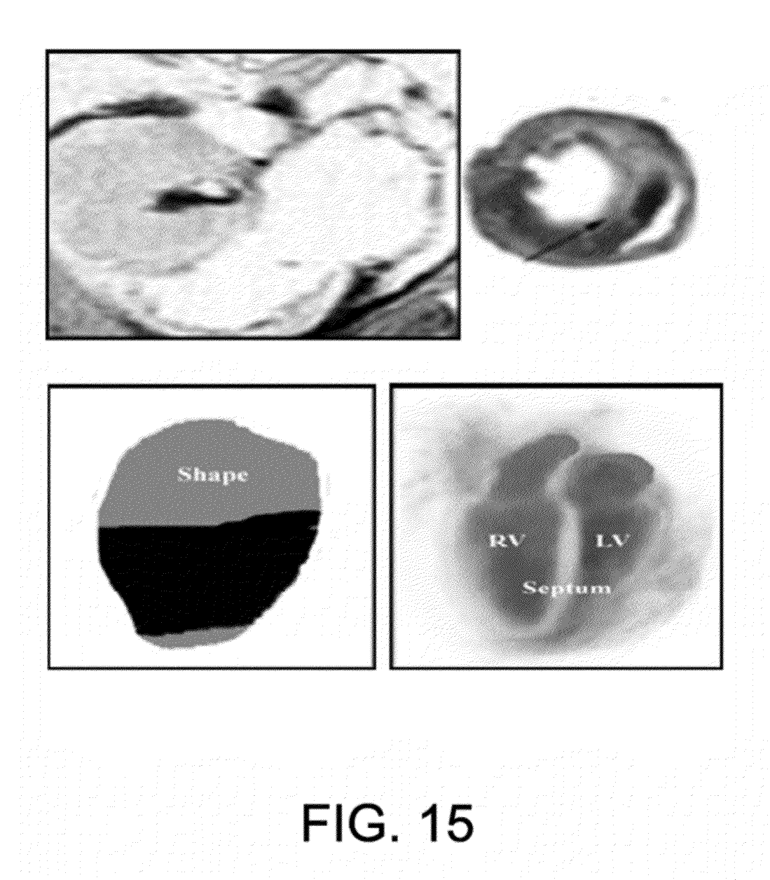

In various embodiments, histologic digital images and/or MRI images can be co-registered by using fiduciary markers or prominent features visible on both histology and/or MRI images. By using a pixel-by-pixel match of different regions in cardiac territories, cardiac mass can be extracted out and shapes of cardiac features can be determined. For example, FIG. 15 illustrates an example Histology-MRI correlation by a point by point match of MRI and histology digital image.

A wide variety of shape analysis may be conducted for various tissue features as desired in embodiments of the invention. For example, a cardiac tissue shape may be determined by intuitive measurements using a hypothesis of compactness, an eccentricity, a rectangularity, statistical shape analysis by spatial configuration variation, and/or deformation analysis by volumetric variation in shape such as feature based methods or variation in position such as geometry based transformation. As shown in FIG. 15, the shape may be approximately equal to the surface area/volume^{2/3}.

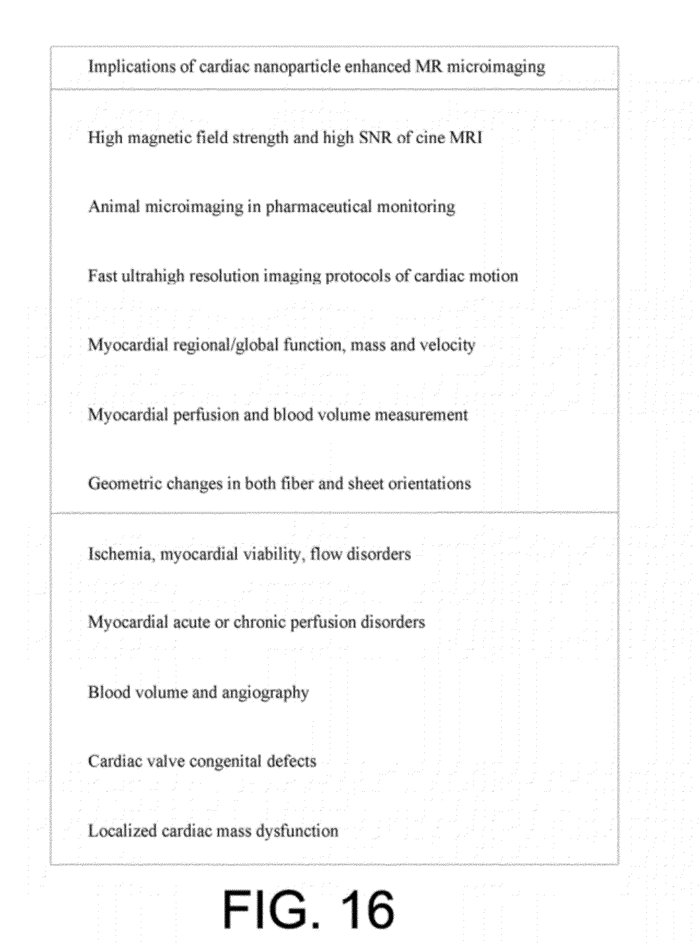

FIG. 16 illustrates an example diagrammatic sketch showing clinical implications of SPIOM enhanced MR microimaging, according to an example embodiment of the invention. As shown in FIG. 16, SPIOM enhanced MR microimaging may facilitate high magnetic field strength MRI imaging, such as 21 Tesla imaging. Additionally, microimaging of various tissue and/or pharmaceutical monitoring may be facilitated. Fast imaging protocols may also be facilitated, such as fast ultrahigh resolution imaging protocols of cardiac. In this regard, various 4D images and/or modeling of tissue may be facilitated. Additionally, images may be generated of myocardial regional/global function along with mass and velocity. Images may further be generated for myocardial perfusion and blood volume and/or geometric changes in both fiber and sheet orientations.

In example embodiments of the invention, whole heart reconstruction of cardiac structure and function by MRI enriched imaging with histology data may provide computation modeling to predict pathophysiological behavior and response to experimental or clinical interventions. Example embodiments of the invention may provide for automated construction of computational mesh aided with atlases and computational visualization and modeling to measure cardiac structures and function. The advanced techniques can be illustrated as workflow from MRI- and histology-based segmentation, to registration of histological sections, co-registration of data sets as probabilistic atlas, and finite element mesh generation to answer computational modeling of cardiac histoanatomy.

Linking cardiac histoanatomy with electromechanical function may pose a risk of spatial heterogeneity in cell properties, misrepresentation of coupling, activation timing from cardiac microstructure. It may need structure-function model development using structural insight and electromechanics with potentials of modeling pathophysiologically disturbed behavior.

Advanced segmentation and tracking of cardiac territories such as a Purkinje network, coronary trees, conduction pathways, and/or simulated sinus node activation patterns will decipher myocardial tissue mechanical and biophysical properties. Other possibilities in accordance with example embodiments of the invention are quantization of branch angles, microstructural fiber sheet arrangement and vessel orientation using efficient finite deformation equations, and/or Navier-Stokes equations for simulation of spatiotemporal distribution of cardiac flow.

According to an example embodiment of the invention, in a beating heart, muscle fiber orientation in different directions and unique lengths may lead to cardiac shape. However, fast algorithms reconstructing 3D histoanatomy of heart in a relatively short processing time may provide clinical utility to support data visualization, interpretation, diagnosis, and prediction of interventions. The computer vision inspires future developments using wavelets, Wold features, and/or fractal analysis as surrogate markers of cardiac diseases in large clinical trials.

Geometric changes in both fiber and sheet orientations may provide a mechanism of radial wall thickening due to myocardial wall shortening. Myocardial shortening may contribute to radial wall thickening and related changes in fiber changes and sheet organization. Predicting the myocyte interaction with extracellular matrix throughout the ventricular wall during myocardial contraction may have implications in detecting abnormalities of a contractile apparatus or extracellular matrix infrastructure

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A method for imaging tissue, the method comprising:
providing polymeric nanoparticles as a contrast agent to subject tissue to be imaged, each polymeric nanoparticle comprising (i) a core that exhibits superparamagnetic properties that enhance tissue imaging when placed within a magnetic field, (ii) an anionic surfacant applied to the core, (iii) a polymeric layer that encapsulates the core and the anionic surfacant, and (iv) an antibody attached to the polymeric layer to facilitate attachment to the subject tissue; and
imaging the subject tissue utilizing a twenty-one Tesla microimaging technique.

2. The method of claim 1, wherein each polymeric nanoparticle comprises a linker protein that attaches the antibody to the polymeric layer.

3. The method of claim 2, wherein the linker protein comprises one of avidin or biotin.

4. The method of claim 1, wherein the core of each polymeric nanoparticle comprises iron oxide.

5. The method of claim 1, wherein the anionic surfacant of each polymeric nanoparticle comprises a fatty acid salt.

6. The method of claim 1, wherein the polymeric layer of each nanoparticle comprises one of polyethylene, polyamide, polycarbonate, polyalkalene, polyvinyl ether, polyglycolide, cellulose ether, polyvinyl halide, polyglycolic acid, or polylactic acid.

7. The method of claim 1, wherein the antibody of each nanoparticle comprises one of antimyoglobin or antitroponin.

8. The method of claim 1, wherein a diameter of each nanoparticle is between about 10 nanometers and about 30 nanometers.

9. The method of claim 1, wherein the polymeric nanoparticles are configured to bind to one of myoglobin or troponin associated with the subject tissue.

10. A method for imaging tissue, the method comprising:
providing a polymeric nanoparticle comprising (i) a superparamagnetic core (ii) an anionic surfacant applied to the core, (iii) a polymeric layer that encapsulates the core and the anionic surfacant, and (iv) an antibody attached to the polymeric layer to facilitate attachment to subject tissue;
supplying the polymeric nanoparticle to the subject tissue as part of a contrast agent; and
imaging the subject tissue utilizing a twenty-one Tesla magnetic resonance microimaging technique.

11. The method of claim 10, wherein providing a polymeric nanoparticle comprises providing a polymeric nanoparticle comprising a linker protein that attaches the antibody to the polymeric layer.

12. The method of claim 11, wherein the linker protein comprises one of avidin or biotin.

13. The method of claim 10, wherein the core of the polymeric nanoparticle comprises iron oxide.

14. The method of claim 10, wherein the anionic surfacant comprises a fatty acid salt.

15. The method of claim 10, wherein the polymeric layer comprises one of polyethylene, polyamide, polycarbonate, polyalkalene, polyvinyl ether, polyglycolide, cellulose ether, polyvinyl halide, polyglycolic acid, or polylactic acid.

16. The method of claim 10, wherein the antibody comprises one of antimyoglobin or antitroponin.

17. The method of claim 10, wherein a diameter of the polymeric nanoparticle is between about 10 nanometers and about 30 nanometers.

18. The method of claim 10, wherein the polymeric nanoparticle is configured to bind to one of myoglobin or troponin associated with the subject tissue.

19. A method for imaging tissue, the method comprising:
providing a polymeric nanoparticle comprising a superparamagnetic core and an antibody that facilitates attachment of the nanoparticle to biological tissue;
supplying the polymeric nanoparticle to the subject tissue as part of a contrast agent; and
imaging the subject tissue utilizing a twenty-one Tesla magnetic resonance microimaging technique.

20. The method of claim 19, wherein the antibody comprises one of antimyoglobin or antitroponin.
